(19) United States
(12) Patent Application Publication
(10) Pub. No.: US 2025/0001038 A1
(43) Pub. Date: Jan. 2, 2025

(54) Bioactive Coating for Topical Application, Process for its Preparation and use of the Bioactive Coating

(71) Applicant: GLOBAL NEW SKIN COSMETICS GMBH, Rodgau-Dudenhofen (DE)

(72) Inventors: Dagmar Scherrinsky, Dreieich (DE); Christoph Sonntag, Neuss (DE)

(73) Assignee: GLOBAL NEW SKIN COSMETICS GMBH, Rodgau-Dudenhofen (DE)

(21) Appl. No.: 18/701,226

(22) PCT Filed: Oct. 5, 2022
(86) PCT No.: PCT/EP2022/077716

§ 371 Date: Apr. 13, 2024

(30) Foreign Application Priority Data
Oct. 14, 2021 (DE) 10 2021 126 687.4

(51) Int. Cl. A61L 15/42; A61F 13/00; A61F 13/01

(52) U.S. Cl. CPC A61L 15/425; A61F 13/00987; A61F 13/01017; A61L 2300/252; A61L 2300/414

(57) ABSTRACT

The invention relates to a bioactive pad (1) for topical application in a cosmetic and/or medical treatment with a water-soluble, sponge-like hydrocolloid matrix (10), which consists of a coated hydrocolloid matrix (10) formed from a foamed aqueous solution which has solidified to form an open-pored structure, the solution containing a hydrocolloid polymer material in a concentration of 1 to 10% by weight, in particular 2 to 5% by weight, and a crosslinking agent containing at least one aldehyde group in a concentration of 0.001 to 0.02% by weight, in particular 0.005 to 0.01% by weight for matrix formation.

### FIELD OF THE INVENTION

The invention relates to a bioactive coating for topical application, a bioactive layer composite with a bioactive coating, a method for producing the bioactive coating and a use of the bioactive coating.

### BACKGROUND OF THE INVENTION

Bioactive overlays in general, processes for the production of such overlays and the use of such overlays have been known for a long time.

However, these are usually only used in the exclusively medical field, so that only medical use is known.

In addition, bioactive dressings in the medical field are usually intended for use in the body or in wounds. They are therefore stable over a longer period of time and only dissolve slowly, e.g. over several days or weeks.

The skin is a large sensory organ with several layers that interacts with the environment and sends signals to the brain about touch, pain, vibrations and their positions.

The most important layers of the skin are the dermis and the epidermis. The epidermis is the visible part of the skin, which is the outer layer of the skin. It is very active with regard to the formation of new skin cells and their slow desquamation. There are different types of epidermal cells.

Due to environmental influences, ageing and internal factors, the activity of forming new skin cells decreases with a person's age, so that the skin becomes more sensitive with age.

The skin can show increasing damage with age, which can also be described as chronic damage. Such chronic damage manifests itself, for example, in dry areas, wrinkles, cracks and thinning of the skin. In addition to chronic damage, the skin can also be damaged by injuries of various kinds, amongst others such as inflammation, radiation, scars etc.

The process of skin healing in the case of injuries is very complicated. Normally, the epidermis (the outer layer) and the dermis (the inner or deep layer) are sealed-off from the environment. The skin forms a protective and stable barrier. As soon as the barrier is damaged, the normal (physiological) recovery process of the skin begins.

In conventional skin healing, a distinction is usually made between four phases:
(1) Hemostasis
(2) Inflammation,
(3) Proliferation, and
(4) Redesign.

However, hemostasis as a phase of healing is disputed among experts. In order to repair the damage caused by an injury, a large number of complex biochemical processes take place after the skin is injured.

After a trauma or injury, blood platelets (thrombocytes) form at the site of the wound within a short time period to form fibrin blood clots. This blood clotting prevents active bleeding (hemostasis).

In the inflammatory phase, foreign bodies in the wound are removed. In the proliferative phase, the factors that cause the cells to divide and migrate are released. The proliferative phase is characterized by angiogenesis, collagen accumulation, the formation of granulation tissue, epithelialization and contraction of the wound.

During angiogenesis, the new blood vessels are formed by the vascular cells of the endothelium.

During fibroplasia and the formation of granulation tissue, the fibroblasts, collagen and fibronectin produce a new extracellular matrix (ECM), At the same time, epithelialization of the epidermis takes place. The epithelial cells become productive and prepare a layer for the new tissue.

Stenosis occurs and the wound becomes smaller due to the movement of the fibroblasts. This occurs when the fibroblasts move to the wound surface. The cells contract at the edges in a mechanism similar to that of smooth muscle cells.

During the maturation and remodeling phase, the collagen forms again and accelerates along the tension lines. The cells that have fulfilled their task and are no longer needed die (apoptosis).

This process is complicated and also prone to errors. It is, therefore, possible that a poorly healing area may result.

In the past, lipid and moisturizing creams were used to prevent this. However, it has been found that the use of such creams and ointments on damaged skin is not sufficient

However, the majority of bioactive coatings known in the prior art are not suitable for purely external and non-invasive application to the skin, which is also referred to as topical application, where the bioactive coating with its components is applied to the skin and additives are absorbed or resorbed by the skin as quickly and completely as possible (especially for intensive skin care).

The materials most likely to be used for wound care at the time of application are hydrocolloids such as gelatine. These are often incorporated into plasters, whereby the plasters are often intended for long-term use.

Such plasters are taught, for example, by the publications WO 1994/17137 A1, US 2012/0059301 A1 and US 2011/0257617 A1. Hydrocolloid compositions are also known, for example, from WO 2007/048193 A1, WO 2004/080500 A1 and US 2012/0209229 A1 is known, For example, DE 10 2013 111 322 A1 teaches a sponge made from a Hydrocolloid polymer material, which can be described as a bioactive overlay and can be implanted in the body as a wound or burn implant.

When used in the body or in wounds, it is intended that the bioactive overlay remains in the form of a sponge over a long period of time and only decomposes slowly, i.e. over days or weeks, and is absorbed by the body, which, however, is disadvantageous in the case of topical application, since the bioactive pad may contribute to the care and/or healing of the skin, but should not remain on the skin for a long period of time for reasons of practicability. Further, these disclosures relate exclusively to medical use and not to cosmetic use.

In summary, intensive skin care as part of cosmetic treatments is necessary to prevent dry areas, wrinkles and the development of chronic damage and to support skin regeneration and maintain the skin's healthy structure. This is because the aim is to remove dead skin cells and support the regulation of moisture and elasticity in damaged skin.

In addition to preventing chronic damage, it is also desirable to support the skin's natural healing process and/or prevent the skin from recovering, so that the result of natural healing can be cosmetically improved. For example, the aim is to minimize scarring during regeneration, e.g. of acne damage, and also to improve the healing results of skin damaged by neurodermatitis.

The solution to these tasks is possible by using the prior art solutions because they are either not suitable for topical application and/or not suitable for short-term but intensive treatment or would have to remain on the skin for a long and therefore impractical period of time.

Therefore, the invention is based on the task to overcome the aforementioned disadvantages and to provide a bioactive coating in form of a bioactive pad as well as a method for its production and its use, wherein the bioactive coating or bioactive pad is to be usable topically for the intensive care of the skin, is to be absorbable by the skin in a short time and is to care for the skin.

### SUMMARY OF THE INVENTION

This task is accomplished by the combinations of features according to the main claim and in accordance with the secondary claims.

According to the invention, a bioactive pad for topical application in a cosmetic and/or medical treatment is proposed. The bioactive pad has a water-soluble, sponge-like hydrocolloid matrix, formed from a foamed aqueous solution that has solidified into an open-pored structure. The aqueous solution is preferably based on sterile water. In particular, an open-pored or open-cell foam can be formed by foaming the still liquid solution, which can then be retained and solidified in its open-pored or open-cell structure, for example, by freezing and in particular, by shock freezing.

Accordingly, the structure and thus the hydrocolloid matrix preferably has pores or cells. During foaming, the solution can significantly increase its volume, so that the open-pored structure (foam) or the hydrocolloid matrix can have seven to eight times the volume of the solution. For example, 3.78 to 4.32 liters of foam can be produced from 540 ml of the solution by foaming. According to the invention, it is envisaged that the solution, i.e. the still liquid solution before foaming and before solidification to form the hydrocolloid matrix contains a hydrocolloid polymer material in a concentration of 1 to 10% by weight, in particular 2 to 5% by weight, more particularly 2 to 3% by weight, and a crosslinking agent containing at least one aldehyde group in a concentration of 0.001 to 0.02% by weight, particularly preferably 0.001 to 0.02% by weight, or 0.005 to 0.01% by weight for matrix formation. In relation to the crosslinking agent, a concentration of 0.005 to 0.01% by weight has proved to be particularly advantageous, as this allows a particularly good water solubility of the hydrocolloid matrix or foam to be achieved with simultaneously good foamability of the solution. It should be emphasized that the percentages by weight preferably refer to the concentration in the solution that has not yet solidified into the open-pored structure and not to the concentration in the solidified structure or the hydrocolloid matrix, which may differ. This very small amount of crosslinking agent in the solution leads to a very small amount of crosslinking agent in the solidified structure or in the hydrocolloid matrix, which is particularly advantageous, as this results in a very good water solubility of the hydrocolloid matrix or the bioactive coating of the bioactive pad, whereby the hydrocolloid matrix and in particular the bioactive coating in form of the bioactive pad can be applied to a skin within a short time and preferably within a few minutes (10 minutes) and preferably within one minute on the skin and in particular within a maximum of 60 minutes is essentially completely absorbed by the skin, which is necessary or at least extremely advantageous for topical treatment.

The solidified structure or the hydrocolloid matrix formed by it can be also be described as a sponge because of its open-pored structure. Since the solidified solution is open-pored or open-cell and in particular, sponge-like, the hydrocolloid matrix itself can also be described as open-pored or open-cell and sponge-like and having the pores.

As already explained, the solution solidifies after foaming to form an open-pored structure with corresponding pores. As a result, the hydrocolloid matrix also has pores. In a particularly advantageous variant of the invention, it is provided that the pores or cells have a diameter of less than 1.0 mm, preferably less than 0.5 mm. Preferably, this applies to all pores or cells of the hydrocolloid matrix. Since the pores or cells do not necessarily have to be spherical, but can also be honeycomb-like, the diameter is defined as the average, largest or smallest diameter or distance between opposing pore/cell walls of a respective pore or cell understood. Accordingly, the solution can be foamed until the pores, and in particular all pores, have such a diameter.

Furthermore, an advantageous design of the bioactive coating of the bioactive pad provides that the hydrocolloid matrix, and in particular the solution from which the hydrocolloid matrix is formed, contains ceramides as an additive which are homogeneously distributed in the hydrocolloid matrix and embedded in it. The ceramides serve to protect the skin from drying out and minimize itching. Preferably, it is provided that the ceramides without mediators, by which dispersion in the still liquid, aqueous solution is to be facilitated, and in particular without neutral oil, can be added to the still liquid solution and are dispersed in it so that the ceramides can be homogeneously distributed in the solution without reducing the foaming properties of the solution.

Furthermore, the solution can be foamed and the foamed solution can be placed in molds before solidification, so that the foamed solution solidifies in a plate-like form during solidification, resulting in a plate-like hydrocolloid matrix and correspondingly a plate-like hydrocolloid matrix bioactive layer can be formed. The plates can be between 10 and 50 mm thick, in particular 35 mm thick, and can be divided into formats suitable for the application, such as 7.5 cm×4.0 cm×0.35 cm.

Alternatively, the solidified solution or the hydrocolloid matrix can also be decomposed and, in particular, crushed or ground so that the hydrocolloid matrix is present in powder form as a hydrocolloid matrix powder (“matrix powder”) and the bioactive coating is in powder form or can be used in this powder form.

In particular, the hydrocolloid matrix or the foamed and as an open-pore structure solidified solution is absorbable due to the open-pore structure and the hydrocolloid matrix formed by the hydrocolloid polymer material make the solidified solution biomimetic and is thus particularly well absorbed by the skin to which it is applied.

The hydrocolloid polymer material can be a natural polymaterial or an artificial polymeric material. Preferably, the hydrocolloid polymer material is a collagen or a gelatine or a gelatine crosspolymer.

Collagen and gelatine are proteins. Gelatine is produced by hydrolysis of collagen. Depending on the starting material (such as pork, calf or bovine skin) and, by hydrolysis with alkali or acids, gelatine types with different protein structures and different foaming and resorption behavior are produced. High-molecular proteins of collagen can cause foreign body reactions in the skin upon contact.

This risk is significantly lower with the comparatively low-molecular proteins in gelatine. Gelatine is therefore preferably used as the material for the hydrocolloid matrix. Another preferred hydrocolloid polymer material is bovine gelatine with a Bloom value of at least and preferably more than 250 Bloom.

The hydrocolloid polymer material or the gelatine cross-polymer forms the foam base, through which the structure is formed with the help of the cross-linking agent, in which the ceramides and, if necessary, other components/additives of the bioactive coating are embedded. For intensive care of the skin, further additives or components can also be added to the aqueous solution and preferably homogeneously distributed or dispersed so that the other additives are also homogeneously distributed in the hydrocolloid matrix and embedded in it after the aqueous solution has foamed and solidified. For this purpose, the further additives can also be dissolved in preferably sterile water, so that a second aqueous solution is formed, whereby the second aqueous solution can then be added to the first aqueous solution described above and dispersed therein.

In addition to ceramides, additives and ingredients include growth factors and peptides, especially silk peptides, which can be used for the intensive care of wrinkled skin damaged by environmental factors and skin stressed by chronic skin diseases such as psoriasis and neurodermatitis.

The bioactive overlay is preferably designed to be able to completely dissolve on the skin, whereby the hydrocolloid matrix and/or the additives embedded in it are preferably taken up by the skin, i.e. absorbed. Especially when antibacterial and/or antiseptic additives are provided, adding ceramides is particularly advantageous, as these counteract drying out of the skin due to the antibacterial and/or antiseptic additives. As mentioned, when adding ceramides, it is advantageous if these are homogeneously distributed in the still liquid solution by dispersion, in particular pure and/or without agents that facilitate dispersion, i.e. in particular without an oil and further in particular without neutral oil. The ceramides are dispersed during dispersion or microdispersion.

The ceramides are homogeneously distributed in the still liquid solution by stirring, for example, by a planetary agitator, before the solution is foamed and solidified, so that the ceramides are homogeneously distributed in the solidified solution after solidification.

Dispersion can also take place in several stages, whereby the term “several stages” is understood, that the ceramides are first distributed in the solution at a first speed for a first period of time (coarse) and then at a higher speed than the first speed for a second period of time.

If the ceramides are not added in pure form but, added with neutral oil, the solution with the increase concentration of cross-linking agents is needed, as more cross-linking agent would be necessary for good foamability, which would mean that the hydrocolloid matrix would no longer be readily watersoluble and therefore no longer suitable for rapid absorption into the skin or topical application, for example in the context of intensive care.

For the care or intensive care of the skin, various other additives can be added to the liquid or possibly already partially cross-linked, i.e. partially foamed, solution and distributed in it so that they are later embedded in the hydrocolloid matrix or in the open-pored structure.

According to an advantageous further development of the bioactive coating of the bioactive pad, it can be provided accordingly that the hydrocolloid matrix and in particular the solution has at least one of the following active components as an additive, which is preferably added to the still liquid solution and can be homogeneously distributed in it before foaming and solidification: Epidermal growth factors EGF (Epidermal Growth Factor, fibroblast growth factors FGF (Fibroblast Growth Factor, insulin-like growth factors IGF (Insulin-like Growth Factor, silk peptides, copper tripeptides, hexapeptides and octapeptides, such as acetyl octapeptide and octapeptide-8.

The bioactive overlay is intended to provide an effective, combined intensive care material with antibacterial properties for skin that has been attacked and stressed by environmental factors, ageing or chronic skin diseases. Infection is a major problem in intensive care of damaged skin. As a solution for this, the bioactive overlay can, for example, have an antibacterial effect due to silk peptides. Compared to other antibacterial additives known in the prior art, silk peptides have the advantage that the silk peptides not only have an antibacterial effect and thus protecting against infections, but also protect the skin from damage. The silk peptides do not dry out and therefore do not delay the healing or regeneration of the skin.

In the case of chronically damaged or stressed skin caused by neurodermatitis, psoriasis or acne, in which the regeneration of the affected skin is delayed or does not occur, it is particularly beneficial if additives in the bioactive overlay that support the cell activity of the skin. If the cell activity of damaged skin is supported, less scar tissue is formed, which reduces the risk of scarring which has a corresponding cosmetic effect. An epidermal growth factor, which can be added to the solution as an active component or additive, is particularly suitable for this purpose.

In general, growth factors promote cell growth with regard to support of tissue regeneration.

In addition, the hydrocolloid matrix and in particular the solution from which the hydrocolloid matrix is formed may contain glutaraldehyde and/or coco-amidopropyl betaine (CAPB) as an additive or as the crosslinking agent. CAPB can also be used to increase the resistance of the bioactive matrix so that it does not disintegrate too quickly on the skin.

If CAPB is used, according to a particularly advantageous further embodiment it is provided that it is dosed in such a way that the CAPB is present in the still liquid solution in a concentration of 0.005 to 0.01% by weight or 1 to 3% by weight in order to induce or promote foam formation.

Accordingly, CAPB improves the quality of the foam and increases the quantity of bubbles. CAPB also serves to moisturize the skin and reduce irritation. CAPB also has an antiseptic effect.

The same applies to glutaraldehyde or a glutaraldehyde solution in a concentration of 0.001 to 0.01% by weight and in particular 0.005% by weight in the still liquid solution.

If CAPB and antibacterial silk peptides are added to the solution or the hydrocolloid matrix, it has been shown that the antibacterial effect of the bioactive coating is surprisingly stronger than the antibacterial effects of the individual components (CAPB and silk peptides) as expected. The antibacterial effect of the silk peptide is thus enhanced by the CAPB.

Accordingly, in a particularly advantageous further development, both silk peptides and CAPB are used as additives to the solution, whereby a particularly good antibacterial effect can be achieved.

It is preferable that the bioactive overlay consists exclusively of components/additives that can be absorbed by the skin and in particular is designed to disintegrate substantially completely on a skin on which the bioactive layer is placed or applied within a period of 10 minutes, in particular 5 minutes, further in particular one minute, and in particular to be removed from the skin within a period of maximum 120 minutes, preferably 60 minutes on which skin the bioactive overlay is applied topically. The abovementioned additives also form components of the bioactive overlay. Preferably, the foam-like hydrocolloid matrix with the additives embedded therein disintegrates accordingly so that they can be absorbed by the skin specifically at the site of application.

In order to prevent the bioactive coating or the hydrocolloid matrix or the components or additives embedded therein from causing a foreign body reaction, these are preferably biomimetic and non-toxic.

According to one aspect of the invention, the bioactive coating of the bioactive pad according to the invention is suitable or intended for use in a medical treatment, in particular a topical treatment of the skin, in particular the treatment of external wounds and/or skin irritations. Due to the high water solubility, the bioactive layer should not or cannot remain on the skin for a long period of time, for example days or weeks.

The bioactive pad containing bioactive coating should not be left on the skin, but should be supplied with additives that support healing in a short time, for example within 120 minutes, preferably 60 minutes.

Accordingly, according to an advantageous variant, the bioactive overlay is intended for use in the treatment of neurodermatitis and/or psoriasis.

In addition, a further aspect of the invention is that the bioactive pad can be used or is used in a cosmetic treatment of skin, in particular a cosmetic treatment of chronically damaged skin, the cosmetic treatment being in particular a topical, i.e. external and local application to the skin.

Cosmetic treatment also includes, in particular, the care or intensive care of chronic damage, which also includes damage caused by psoriasis or neurodermatitis, for example.

In particular, the bioactive overlay is suitable for use in the medical and/or cosmetic treatment of aged skin, rough skin, skin damaged by environmental influences, i.e. wrinkled skin, dry skin and skin that is prone to dryness, skin damage caused by permanent damage as well as dermatitis, neurodermatitis (atopic dermatitis), psoriasis, ichthyosis vulgaris and acne.

In addition to the bioactive coating proposed according to the invention, the invention also relates in a further aspect to a bioactive layer composite with at least two layers, wherein at least a first layer of the at least two layers is formed from a bioactive coating according to the invention.

Preferably, it is further provided that the bioactive layer from which the first layer is formed contains one of the above-mentioned additives as a first additive. Furthermore, the bioactive layer composite has a second layer of the at least two layers of a bioactive coating according to the invention, which also comprises one of the above-mentioned additives as a second additive.

It is provided that the second additive and the first additive differ in concentration and/or type.

Furthermore, the first and/or second layer can be designed to dissolve completely within a predetermined time.

To put it simply, the bioactive layer composite can be formed from several layers of different additives and/or bioactive coatings with different concentrations of the additives, so that the sequence, concentration (quantity and duration in which and with which the additives act on the skin) can be controlled.

Such a bioactive layer composite can also be intended for use in a medical and/or cosmetic treatment. It also applies to the layered composite that it is particularly suitable for the treatment of aged skin, rough skin, skin damaged by environmental influences, i.e. wrinkled skin, dry skin and skin caused by permanent damage, skin damage as well as dermatitis, neurodermatitis (atopic dermatitis), psoriasis, ichthyosis vulgaris and acne.

Furthermore, one aspect of the invention also relates to a method for producing a bioactive overlay and, in particular, the bioactive overlay according to the invention. As already mentioned, the bioactive overlay to be produced as described above, has a biomimetic hydrocolloid matrix, in particular in the form of a sponge. The method of manufacture comprises at least the following steps:
a. Addition of a predetermined quantity of hydrocolloid polymer material to a predetermined quantity of, in particular, sterile water and dispersion of the hydrocolloid polymer material. The hydrocolloid polymer material in the water to form an aqueous solution, the predetermined amount of the hydrocolloid polymer material and the predetermined amount of water being so selected, that the hydrocolloid polymer material is present in the solution or the hydrocolloid matrix at a concentration of 2 to 5% by weight;
b. Single-stage or multi-stage addition of a predetermined amount of a crosslinking agent containing at least one aldehyde group into the aqueous solution and dispersing the crosslinking agent in the aqueous solution, the predetermined amount of crosslinking agent being selected such that the crosslinking agent is present at a concentration of 0.05 to 0.1% by weight in the solution or hydrocolloid matrix;
c. Foaming of the aqueous solution with the crosslinking agent dissolved in it. The solution is at least partially in a foamed, open-pored structure;
d. Freezing and in particular shock freezing of the foamed, open-pored structure so that the foamed, open-pored solution solidified by freezing forms the hydrocolloid matrix.

Furthermore, a bioactive coating of a bioactive pad is preferably obtainable by this method. The single- or multi-stage and, in particular, two-stage addition of the crosslinking agent is understood to mean that the entire crosslinking agent can be added to the aqueous solution in one step or it is alternately possible to first add a first partial quantity of the crosslinking agent and only add a second or the remaining partial quantity of the crosslinking agent after adding and dispersing any ceramides and/or additives present.

An advantageous embodiment of the method also provides that the ceramides are added to the aqueous solution before foaming and homogeneously distributed in the solution by dispersing, the dispersing being carried out in particular by multistage mechanical stirring.

For example, the ceramides can first be distributed in the aqueous solution for 2 minutes at a rotational speed of between 500 and 1500 revolutions per minute and then for 3 minutes at a rotational speed of between 3000 and 4000 revolutions per minute. According to an advantageous further development, it is particularly advantageous if the ceramides are added to the solution directly and without a mediator, i.e. pure and in particular without oil or neutral oil, and in particular are dispersed in the solution exclusively by mechanical stirring and preferably by multi-stage mechanical stirring, i.e. homogeneously dispersed.

In order to prevent the at least partially foamed solution from disintegrating again before freezing, for example due to heat generated during stirring, it is also preferably provided that the solution or the foamed, open-pored structure is at least partially foamed during the foaming process and preferably cooled down to freezing and/or kept at a maximum temperature of 35° C., preferably 32° C.

After freezing, it is preferable to dry the solidified solution or hydrocolloid matrix using dry freezing technology. For this purpose, the hydrocolloid matrix can, for example, simply be dried over several hours or days in the device for freezing the hydrocolloid matrix.

By foaming the solution, the foamed open-pored structure formed in this way can also be introduced specifically into molds for freezing and, in particular, into pre-cooled molds which are at least cooled to such an extent that the foamed structure does not disintegrate due to the temperature of the molds, whereby not the entire (liquid) solution has to be foamed and correspondingly not the entire (liquid) solution has to be transferred into the solidified solution.

As the end product is applied to the affected or damaged skin, the bioactive dressing should be sterile and pyrogen-free in order to reliably rule out infections or foreign body reactions. For this purpose, the sponge or hydrocolloid matrix is preferably produced in all processes. The products are manufactured and, if necessary, assembled as contamination-free as possible.

For example, sterilized water can be used as the starting base for the aqueous solution. Furthermore, dispersion in a closed agitator system can also be used to ensure that the water is not dispersed during the production essentially no germs are introduced. Additionally or alternatively, however, a further process step may also be provided, according to which the hydrocolloid matrix or the solidified structure is sterilized and, in particular, sterilized by irradiation with gamma rays at a suitable dosage.

In addition to the bioactive coating of the bioactive pad per se, the application of bioactive hydrocolloid materials or the application of the bioactive coating proposed according to the invention with the hydrocolloid matrix, which is essentially formed by such a bioactive hydrocolloid material (hydrocolloid polymer material), is—as far as is known—also known in cosmetics or in cosmetic treatments, such as the intensive care of damaged and wrinkled skin. The cosmetic use is characterized in particular by the fact that the application of the bioactive pad is exclusively topical (external) and non-invasive. Furthermore, the cosmetic application is characterized in particular by that the healing of a wound or skin wound should not primarily be accelerated. Rather, chronic damage in particular should be treated or the natural healing of the skin should be supported in such a way that the skin is healed faster.

The result of the healing is visually appealing, for example, no scars or reduced scars are formed during healing.

Accordingly, a further aspect of the invention relates to the use of a bioactive coating in form of a bioactive pad according to the invention and/or a bioactive coating obtainable by the method according to the invention for the cosmetic treatment of skin.

Skin damages that can be treated cosmetically are in particular aged skin, rough skin, skin damaged by environmental influences, i.e. wrinkled skin dry skin and skin damage caused by permanent damage as well as dermatitis, neurodermatitis (atopic dermatitis), psoriasis, ichthyosis vulgaris and acne.

In the context of the particularly cosmetic use of the bioactive coating of the bioactive pad, it can be moistened before being applied to the skin and/or the skin can be prepared with a peeling or micro-needling.

Once the bioactive overlay has been applied, the hydrocolloid matrix forms a guide rail for forming collagen cells. The molecules of the hydrocolloid matrix and the additives embedded in it show their effect on cellular collagen cells level and help to control the bacterial population and regenerate the skin.

The features disclosed above can be combined as desired, provided this is technically possible and they do not contradict each other.

### BRIEF DESCRIPTION OF THE DRAWINGS

Other advantageous embodiments of the invention are characterized in the sub-claims or are shown in more detail below together with the description of the preferred embodiment of the invention with reference to the figures.

The figures show:

FIG. 1 bioactive overlay;

FIG. 2 manufacturing process for a bioactive pad containing bioactive coating.

The figures are schematic examples. Identical reference signs in the figures indicate identical functional and/or structural features.

### DETAILED DESCRIPTION OF PREFERRED EMBODIMENTS

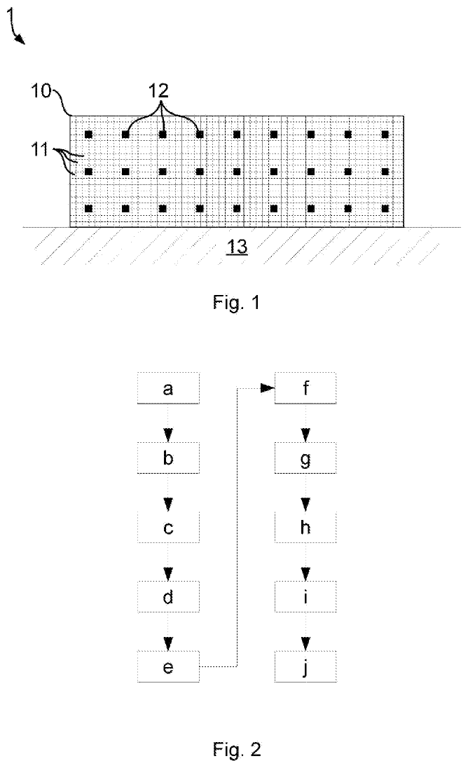

FIG. 1 shows an exemplary bioactive overlay 1, which is essentially characterized by the fact that it has a water-soluble, sponge-like hydrocolloid matrix 10, which is formed from a foamed aqueous solution that has solidified to form an open-pored structure, which accordingly has a large number of pores 11.

FIG. 1 shows an example of this on a skin 13 for topical cosmetic treatment. Treatment applied.

The aqueous solution, which is foamed and converted into a rigid form or structure, for example by shock freezing, is produced by mixing a hydrocolloid polymer material and a crosslinking agent containing at least one aldehyde group with water, particularly sterile water, to form a matrix. However, it is important to achieve both a good foaming or foaming behavior of the solution as well as a high water solubility of the structure created by foaming and solidified by freezing, so that the bioactive pad containing bioactive coating 1 can be quickly applied to the skin 13 on which it is topically applied and can be absorbed by the skin with any additives it may contain.

For this purpose, the hydrocolloid polymer material is added to the (still liquid) solution or to the water as the starting base for the solution in a concentration such that the hydrocolloid polymer material is present in the solution in a concentration of 2 to 5% by weight.

It is particularly important that only very little cross-linking agent is added to the base solution formed from the water and the hydrocolloid polymer material, as otherwise the water solubility and thus the resorbability of the resulting hydrocolloid matrix would be too poor for a practicable topical application of the bioactive coating. Accordingly is provided that the crosslinking agent is added to the (still liquid) solution in an amount such that it is present in the solution in a concentration of 0.005 to 0.01% by weight.

Ceramides 12 have also been added to the bioactive coating 1 of the bioactive pad shown, which effectively prevent the skin 13 from drying out in the area of the application of the bioactive overlay. In addition to ceramides 12, the bioactive coating 1 may also contain other additives and in particular growth factors and/or silk peptides as additives. The ceramides 12 are preferably added to the solution in pure form and dispersed in the solution purely mechanically and in particular by stirring and thereby are homogeneously distributed in the solution so that they are also homogeneously distributed in the hydrocolloid matrix 10 and embedded in the structure. Various known stirrers can be used for this purpose, whereby it has been shown that the stirring speed should not be too high. Thus, stirring can also be carried out in the simplest way, simply mechanically with a stirring object.

It is particularly advantageous here that the ceramides 12 are added to the (still liquid) solution pure and without a mediator, such as neutral oil and they are homogeneously distributed in the solution by purely mechanical stirring, for example.

This maintains the good foamability of the solution and at the same time achieves good water solubility of the solidified structure, which would otherwise be negatively affected by, for example, oil or neutral oil would be influenced

FIG. 2 shows the schematic sequence of an exemplary process for the production of a bioactive pad containing bioactive coating 1, whereby, for example, the bioactive coating 1 according to FIG. 1 can be produced.

According to the process illustrated in FIG. 2, the production is carried out with at least the following steps, in the following order. The equipment required for this is conventional equipment. If no further information is given on individual measures, suitable and typical values can be used, e.g. stirring can be carried out at a speed of approx. 200-500 rpm. for a period of up to approx. 20-25 minutes (unless otherwise specified):
a. Adding a predetermined amount of bovine gelatine with a Bloom value greater than 250 Bloom to a predetermined amount of sterile water and dispersing or stirring the gelatine in or with the sterile water to form an aqueous solution, wherein the predetermined amount of bovine gelatine and the predetermined amount of sterile water are selected such that the bovine gelatine is present in the solution at a concentration of 2 to 5% by weight, wherein the dispersing is carried out by stirring in a closed planetary stirring system until the bovine gelatine is completely dissolved or homogeneously distributed in the sterile water, and an aqueous solution was formed, which can also be referred to as a gelatine solution;
b. Adding a predetermined amount of ceramides 12 to the aqueous solution, wherein the ceramides 12 are added pure and in particular without a neutral oil is added to the aqueous solution and dispersed in it, wherein the dispersing is carried out by stirring in the closed planetary stirring system until the ceramides 12 are homogeneously dispersed in the aqueous solution and the aqueous solution is dispersed as an aqueous solution, gelatine-ceramide solution;
c. Adding a first partial amount of a predetermined amount of glutaraldehyde as crosslinking agent to the aqueous solution, which can be designated as gelatine-ceramide solution, and dispersing the crosslinking agent in the aqueous solution, wherein the predetermined amount of glutaraldehyde is added to the aqueous solution of the glutaraldehyde is selected so that the glutaraldehyde is present in the solution at a concentration of 0.005 to 0.01% by weight, dispersion is carried out by stirring in the closed, planetary stirring system until the glutaraldehyde is completely dissolved or homogeneously distributed in the aqueous solution, whereby macromolecules of the aqueous solution are crosslinked by the glutaraldehyde during dispersion and a first degree of crosslinking is achieved and wherein the aqueous solution with the glutaraldehyde can be designated as a gelatine-ceramide-glutaraldehyde solution;
d. Cooling the aqueous solution or the gelatine-ceramide-glutaraldehyde solution with continuous stirring in the planetary stirring system to a predetermined temperature of preferably 32° C. and maintain the temperature at least during the following steps e to g;
e. Adding and dispersing additives into the gelatine-ceramide-glutaraldehyde solution with continuous stirring, whereby the addition and dispersion of the additives is carried out by means of a stirrer can be dissolved in a second aqueous solution, which is added to the gelatine-ceramide-glutaraldehyde solution and distributed homogeneously therein;
f. Adding a second partial amount of the predetermined amount of glutaraldehyde, the first and second partial amounts in total being of the predetermined amount of glutaraldehyde;
g. foaming of the gelatine-ceramide-glutaraldehyde solution with the additives contained therein, by means of preferably multi-stage stirring in the closed, planetary stirring system, so that the gelatine-ceramide-glutaraldehyde solution is foamed. The product achieves a second degree of crosslinking, which is higher than the first degree of crosslinking, and is present as a foamed open-pored structure (foam) whose pores have a diameter of preferably less than 0.5 mm;
h. Insertion of the foamed, open-pored structure or the foam into at least one pre-cooled mold;
i. Freezing and in particular shock freezing of the foamed, open-pored structure in the at least one mold, so that the structure solidifies and thus forms the hydrocolloid matrix 10;
j. Drying of the solidified structure or of the foamed and formed into a aqueous solution or the hydrocolloid matrix 10 solidified into an open-pored structure, wherein the hydrocolloid matrix 10 can remain in a device for freezing according to step I for drying;

In steps a) to c), homogeneous dissolution can be assessed either by a measuring method or, alternatively, by visual inspection. If the homogeneity is insufficient, the stirring process can be repeated.

During foaming, the volume of the aqueous solution can be increased by a factor of 7 to 8 in particular due to the good foaming behavior, so that the foam or later the open-pored, solidified structure is present with 7 to 8 times the volume of the solution.

The increase in volume should preferably be in this range, but can also be less. If the volume change is significantly too low or too high, the pore size in the bioactive layer may change.

For example, 15 g of bovine gelatine A with a Bloom value of >250 can be added to 500 ml of sterile water at 1500 rpm and stirred in a closed stirring system (e.g. Kennwood stirring system) with a planetary mixer.

The ceramides are dissolved in a stirrer at 55° C. for 5 minutes. Then 15 mg of ceramides are added and stirred again for 2 minutes at 1000 rpm and dispersed for a further 3 minutes at 3500 rpm. To the homogeneous aqueous gelatine-ceramide solution, 10 mg of glutaraldehyde is added as a crosslinking agent and crosslinked at 3000 rpm to achieve a high degree of efficiency.

This gelatine-ceramide-glutaraldehyde solution is cooled down to approx. 30° C.-32° C. in 25 minutes with continuous, planetary stirring at approx. 500 rpm. This temperature is maintained until the end of foam production.

As soon as the homogeneous water-glutaraldehyde-gelatine-ceramide dispersion has cooled to approx. 32° C. with slow stirring at 500 rpm, the peptides, e.g. EGF; FGF, IGF or other peptides are added in the form of 10 ml of an aqueous solution and dispersed for 5 minutes at 3000 rpm to finally mix all components homogeneously. After another 5 minutes of stirring, another 10 mg of glutaraldehyde, dissolved in 3 ml of sterile water, is added and dispersed at 3000 rpm for approx. 5 minutes. Then another 30 minutes at 3500 rpm. A clear foam appears at some point.

After approx. 40-45 minutes total stirring time, reduce the stirring speed from 3500 rpm to approx. 2500 rpm and continue stirring for a further 20 minutes. After the further addition of glutaraldehyde, the developing homogeneous foam has increased to approx. 7 to 8 times the initial aqueous foam volume to 4,200 ml of foam.

The foam is then placed directly into pre-cooled molds and shock-frozen in a deep freezer at −50 to −70° C., for example. Freeze-drying takes place in the pre-cooled freeze dryer over a period of 54 hours at a pressure of up to 0.015 mbar. The dried hydrocolloid matrix 10 can then be made up, i.e. cut up and packaged. For example, the hydrocolloid matrix 10 is cut into 7.5 cm×4.0 cm×0.35 cm matrices, packed in sealed-edge bags and these are sealed.

Alternatively, the hydrocolloid matrix 10 can also be crushed into a powder and the powder portioned and packaged.

Furthermore, a bioactive layer composite with at least two layers can be produced, wherein at least a first layer of the at least two layers is made of a bioactive matrice produced as described above or a powder pressed into shape and applied to a carrier layer, where it is bonded to the carrier layer by suitable (known) means.

Although preferably produced under low-germ conditions and in a closed stirring system, the hydrocolloid matrix 10 or the matrices or the powder can be irradiated with gamma radiation at, for example 17.5 kGy gamma-sterilized to ensure a later, germ-free use.

The invention is not limited to the preferred embodiments described above. Rather, a number of variants are conceivable which also make use of the solution described in fundamentally different embodiments.

1. A bioactive pad (1) designed for topical application in a cosmetic or medical treatment comprising:
a hydrocolloid matrix (10), formed from a foamed aqueous solution that has solidified into an open-pored structure, the hydrocolloid matrix is water-soluble and spongy;
wherein the foamed aqueous solution contains a hydrocolloid polymer material in a concentration of 1 to 10% by weight, and a crosslinking agent containing at least one aldehyde group in a concentration of 0.001 to 0.02% by weight, for the hydrocolloid matrix formation.

2. The bioactive pad of claim 1 wherein the hydrocolloid matrix (10) has a plurality of pores (11) and the plurality of pores (11) have a diameter of less than 1.0 mm, preferably have the diameter less than 0.5 mm.

3. The bioactive pad of claim 1,
wherein the hydrocolloid matrix (10) and in particular the foamed aqueous solution from which the hydrocolloid matrix (10) is formed contains a predetermined amount of ceramides (12) as an additive, which is homogeneously distributed and embedded in the hydrocolloid matrix (10).

4. The bioactive pad of claim 1
wherein the hydrocolloid matrix (10) and in particular the foamed aqueous solution, contains at least one of the following additives, homogeneously distributed in the hydrocolloid matrix (10):
epidermal growth factor EGF, fibroblast growth factor factors FGF, insulin-like growth factors IGF, silk peptides, copper tripeptides, hexapeptides, octapeptides.

5. The bioactive pad of claim 1
wherein the hydrocolloid matrix (10) and in particular the foamed aqueous solution contains a cross-linking agent that includes but is not limited to glutaraldehyde and/or cocoamidopropyl betaine as an additive.

6. The bioactive pad of claim 1
wherein the bioactive pad (1) consists exclusively of components which can be reabsorbed by the skin and in particular is designed to decompose within a period of 10 minutes.

7. The bioactive pad of claim 1 for use in a topical treatment of a user's skin, treatment of external wounds and/or skin irritations.

8. The bioactive pad of claim 1 for use as a in the treatment of neurodermatitis and/or psoriasis.

9. The bioactive pad of claim 1 for use in a cosmetic treatment of the skin, a cosmetic treatment of chronically damaged skin.

10. The bioactive pad of claim 1, wherein the pad comprises at least two layers, a first layer of the at least two layers is formed from the hydrocolloid matrix and at least one additive selected from the group consisting of ceramide, Epidermal growth factor, fibroblast growth factor, insulin-like growth factor, silk peptide, copper tripeptide, hexapeptide, octapeptide, glutaraldehyde, cocoamidopropyl betaine.

11. A bioactive pad of claim 10,
wherein the first layer and a second layer comprises two different additives.

12. A method for producing a bioactive pad (1), comprising at least the following steps:
a. adding a predetermined amount of a hydrocolloid polymer material to a predetermined amount of water and dispersing the hydrocolloid polymer material in the water to form an aqueous solution, wherein the predetermined amount of the hydrocolloid polymer material and the predetermined amount of the hydrocolloid polymer material are dispersed in the water to form an aqueous solution water so that the hydrocolloid polymer material is present in the aqueous solution in a concentration of 1 to 10 wt. %, in particular 2 to 5 wt. %;
b. single-stage or multi-stage addition of a predetermined amount of a crosslinking agent containing at least one aldehyde group crosslinking agent into the aqueous solution and dispersing the crosslinking agent in the aqueous solution, the predetermined amount of crosslinking agent being selected such that the crosslinking agent is dispersed at a concentration of 0.001 to 0.02% by weight, in particular 0.005 to 0.01% by weight, is present in the aqueous solution;
c. foaming of the aqueous solution with the crosslinking agent dissolved therein, so that the aqueous solution is at least partially dissolved in and has a foamed, open-pored structure;
d. freezing of the foamed, open-pored structure so that the foamed, open-pored structure, which has solidified due to the freezing, forms a hydrocolloid matrix (10).

13. The method of claim 12, wherein
a predetermined amount of ceramides (12) is added to the aqueous solution before foaming and homogeneously distributed in the solution by a dispersion, the dispersion being carried out in particular exclusively by mechanical stirring.

14. The method of claim 13,
wherein the predetermined amount of ceramides (12) is added to the solution directly and without a mediator (neutral oil) and dispersed in the solution by stirring.

15. The method of claim 12,
wherein the hydrocolloid matrix (10) is sterilized by irradiation with gamma rays.

16. The method of claim 12, wherein the bioactive pad (1) is used for cosmetic treatment of a user's skin and cosmetic treatment of chronic damage to the user's skin.
